(19) United States
(12) Patent Application Publication
(10) Pub. No.: US 2025/0066285 A1
(43) Pub. Date: Feb. 27, 2025

(54) INTRINSIC LOW DIELECTRIC CONSTANT POLYIMIDE FILM BASED ON NOVEL DIAMINE HAVING -CF3 GROUP AND 1,4-DIISOPRYOPYLBENZENE UNIT

(71) Applicant: PUSAN NATIONAL UNIVERSITY INDUSTRY-UNIVERSITY COOPERATION FOUNDATION, Busan (KR)

(72) Inventors: Chang Sik HA, Busan (KR); Siva Gangi Reddy NAGELLA, Busan (KR); Ji Yun HAN, Busan (KR)

(73) Assignee: PUSAN NATIONAL UNIVERSITY INDUSTRY-UNIVERSITY COOPERATION FOUNDATION, Busan (KR)

(21) Appl. No.: 18/778,758

(22) Filed: Jul. 19, 2024

(30) Foreign Application Priority Data
Aug. 23, 2023 (KR) 10-2023-0110559
Nov. 24, 2023 (KR) 10-2023-0165704

(51) Int. Cl. C07C 211/54; C08G 73/10; C08J 5/18

(52) U.S. Cl. CPC C07C 211/54; C08G 73/1067; C08J 5/18; C07C 2601/16; C08J 2379/08

(57) ABSTRACT

A novel diamine compound and a low dielectric constant polyimide film based thereon are disclosed. The novel diamine compound is represented by a Chemical Formula 1 as set forth below:

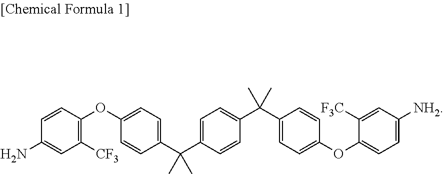 [SMILES: CC(C)(c1ccc(Oc2ccc(N)cc2C(F)(F)F)cc1)c1ccc(C(C)(C)c2ccc(Oc3ccc(N)cc3C(F)(F)F)cc2)cc1]

### CROSS-REFERENCE TO RELATED APPLICATION

This application claims priority to Korean Patent Application No. 10-2023-0110559 filed on Aug. 23, 2023, and Korean Patent Application No. 10-2023-0165704 filed on Nov. 24, 2023, and all the benefits accruing therefrom under 35 U.S.C. 119, the contents of each of which are herein incorporated by reference in their entirety.

### BACKGROUND

### Field

The present disclosure relates to a novel diamine compound and a low dielectric constant polyimide film based on the same.

### Description of Related Art

Polyimide is a highly functional engineering polymer that has good mechanical strength as well as high thermal and chemical stability. Furthermore, polyimide has optical and electrical properties suitable for organic solar cells, organic electroluminescent devices (OLED), flexible displays, wearable electronic devices, and high-frequency wireless communication devices. Therefore, polyimide plays an important role in microelectronics and other applications.

Traditional aromatic polyimide (API) is best suited for this application. However, API has a high water absorption (WU %) due to a polar imide group and has a dark color due to charge transfer interaction (CTI), and thus has low transmittance. API (typically, polyimides based on pyromellitic dianhydride (PMDA) and biphenyl tetracarboxylic acid dianhydride (BPDA)) has insolubility in organic solvents. API has relatively high dielectric constant (Dₖ=about 3.5). For these reasons, API has limitation in in-depth application to the fields of microelectronics and optoelectronics.

Therefore, there is a need for research on changing a molecular structure of the polyimide to improve the solubility, optical, and dielectric properties of the API and to design transparent polyimide with a low dielectric constant.

### SUMMARY

One purpose of the present disclosure is to provide a novel diamine compound with ultra-low dielectric properties, and a low dielectric constant polyimide film based on the same.

In one aspect, the present disclosure provides a diamine compound represented by a Chemical Formula 1 as set forth below:

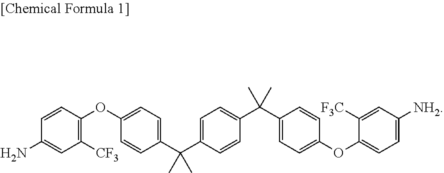 [SMILES: CC(C)(c1ccc(Oc2ccc(N)cc2C(F)(F)F)cc1)c1ccc(C(C)(C)c2ccc(Oc3ccc(N)cc3C(F)(F)F)cc2)cc1]

In one embodiment of the diamine compound, the diamine compound is used for preparing a polyimide precursor.

In another aspect, the present disclosure provides a polyimide precursor prepared by polymerizing a polymerization component including the diamine compound as defined above and at least one kind of acid dianhydride.

In one embodiment of the polyimide precursor, the acid dianhydride is selected from pyromellitic dianhydride (PMDA), biphenyl-tetracarboxylic acid dianhydride (BPDA), 1,2,3,4-cyclobutanetetracarboxylic dianhydride (CBDA), and 4,4′-(hexafluoroisopropyliidene) diphthalic anhydride (6-FDA).

In one embodiment of the polyimide precursor, the polyimide precursor includes a repeating unit represented by a following Chemical Formula 2:

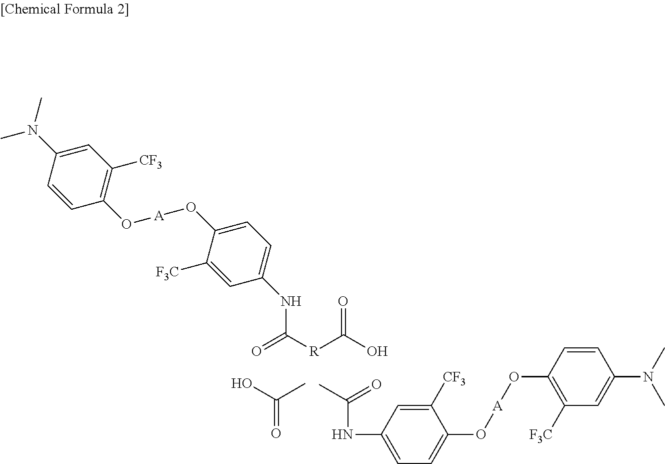

where in the Chemical Formula 2,

A is

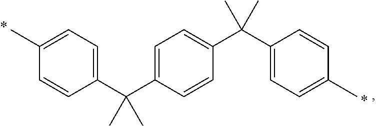

R is a tetravalent organic group,

* is a binding site.

In one embodiment of the polyimide precursor, R is selected from a group consisting of compounds respectively represented by following Chemical Formulas 2-1 to 2-4:

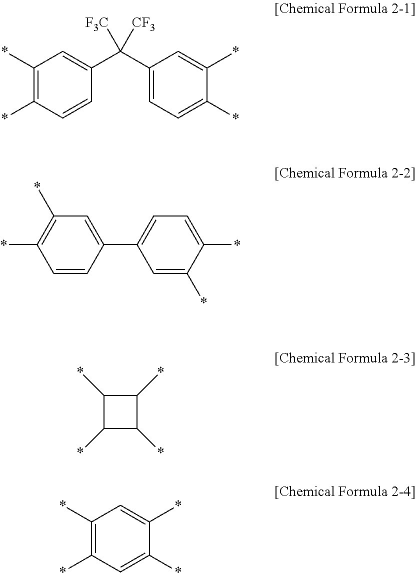 [SMILES: *c1ccc(C(c2ccc(*)c(*)c2)(C(F)(F)F)C(F)(F)F)cc1* ; *c1ccc(-c2ccc(*)c(*)c2)cc1* ; *C1C(*)C(*)C1* ; *c1cc(*)c(*)cc1*]

wherein * is a binding site.

In still another aspect, the present disclosure provides a polyimide film manufactured by applying a composition containing the polyimide precursor as defined above on a substrate and then thermally imidizing the applied composition.

In one embodiment of the polyimide film, the polyimide film includes a repeating unit represented by a following Chemical Formula 3:

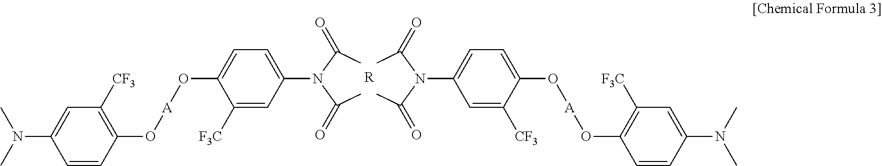

where in the Chemical Formula 3,

A is

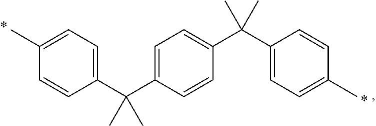

R is a tetravalent organic group,

* is a binding site.

In one embodiment of the polyimide film, R is selected from a group consisting of compounds respectively represented by following Chemical Formulas 2-1 to 2-4:

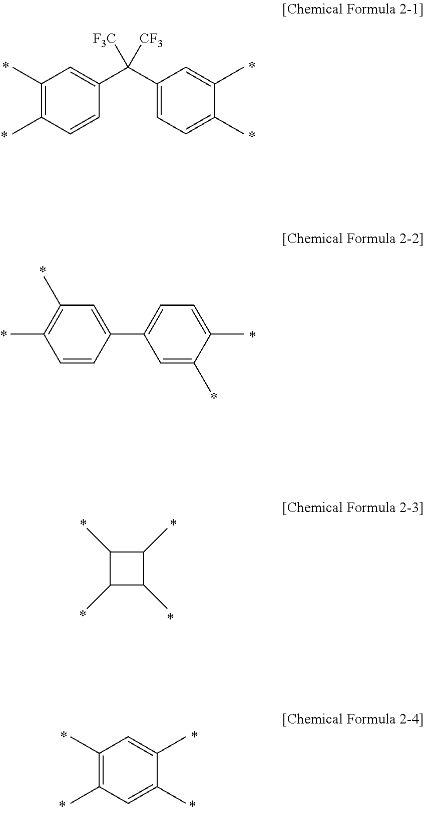

wherein * is a binding site.

In one embodiment of the polyimide film, the polyimide film has a dielectric constant (Dₖ) of 2.11 or lower.

The novel diamine compound represented by the Chemical Formula 1 according to the present disclosure includes a trifluoromethyl group and a 1,4-diisopropylbenzene unit. Specifically, as shown in the 3D molecular structure of the novel diamine compound, the novel diamine compound according to the present disclosure has two 4-(p-phenoxy)-3-(trifluoromethyl)aniline units respectively on both opposing sides of the non-planar center of the 1,4-diisopropylbenzene unit and has a non-planar kinked structure and a skewed structure. When this twisted structure monomer and the dianhydride constitute a polyimide chain, the novel diamine compound may exhibit ultra-low dielectric properties.

The polyimide film synthesized according to the present disclosure has a low dielectric constant (Dₖ) of 2.11 or lower (1.13 to 1.84 at 1 MHz), a high 5% loss temperature (392 to 491° C.), and an appropriate glass transition temperature (Tg) (224 to 254° C.) and has a high tensile modulus (0.827 to 1.076 Gpa), thereby exhibiting excellent heat resistance and thermal stability. Furthermore, the polyimide film synthesized according to the present disclosure has excellent optical properties (T450 up to 83.37%), low moisture absorption (<0.63%), and especially excellent solubility in organic solvents.

As such, the polyimide film of the present disclosure has excellent thermal resistance and thermal stability, optical properties, low moisture absorption, and ultra-low dielectric properties. Thus, the polyimide film of the present disclosure may be applied to interlayer dielectric materials, wearable display devices, etc., and thus has high potential for application in 5G/6G communication applications and the microelectronics industry.

In addition to the effects as described above, specific effects in accordance with the present disclosure will be described together with following detailed descriptions for carrying out the disclosure.

### BRIEF DESCRIPTION OF DRAWINGS

FIG. 1 is a schematic diagram of synthesis of a new diamine compound according to an embodiment of the present disclosure.

FIG. 2 is a 3D molecular structure of a novel diamine compound according to an embodiment of the present disclosure. In this regard, (a), (b), and (c) are projection surface structures in X-, Y-, and Z-directions, respectively.

FIG. 3 shows a synthesis route of a polyimide film based on a novel diamine BPBTAB according to an embodiment of the present disclosure and several commercial dianhydrides.

FIG. 4 is a 3D molecular structure of polyimide based on the novel diamine BPBTAB according to an embodiment of the present disclosure and FDA.

FIG. 5 shows FT-IR spectra of materials synthesized according to an example of the present disclosure.

FIG. 6 and FIG. 7 show 1H NMR spectra of materials synthesized according to an example of the present disclosure.

FIGS. 8 and 9 show 13C NMR spectra of materials synthesized according to tan example of the present disclosure.

FIG. 10 shows a FT-IR spectrum of a polyimide film manufactured according to an example of the present disclosure.

### DETAILED DESCRIPTIONS

Hereinafter, embodiments of the present disclosure will be described in detail with reference to the accompanying drawings. The present disclosure may be subjected to various changes and may have various forms. Thus, particular embodiments will be illustrated in the drawings and will be described in detail herein. However, this is not intended to limit the present disclosure to a specific disclosed form. It should be understood that the present disclosure includes all modifications, equivalents, and replacements included in the spirit and technical scope of the present disclosure. While describing the drawings, similar reference numerals are used for similar components. In the accompanying drawings, the dimensions of the structures are shown to be exaggerated to make the clarity of the present disclosure.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “including”, “include”, and “including” when used in this specification, specify the presence of the stated features, integers, operations, elements, and/or components, but do not preclude the presence or addition of one or more other features, integers, operations, elements, components, and/or portions thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

A diamine compound according to an embodiment of the present disclosure may be represented by a Chemical Formula 1 as set forth below:

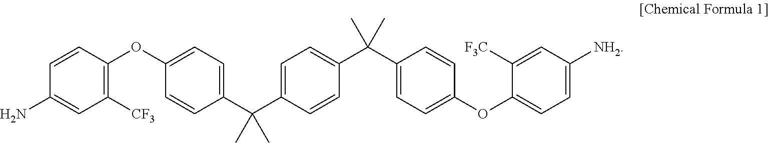 [SMILES: CC(C)(c1ccc(Oc2ccc(N)cc2C(F)(F)F)cc1)c1ccc(C(C)(C)c2ccc(Oc3ccc(N)cc3C(F)(F)F)cc2)cc1]

The novel diamine compound represented by the Chemical Formula 1 includes a trifluoromethyl group and a 1,4-diisopropylbenzene unit. Specifically, as shown in the 3D molecular structure of the novel diamine compound, the novel diamine compound according to the present disclosure has two 4-(p-phenoxy)-3-(trifluoromethyl)aniline units respectively on both opposing sides of the non-planar center of the 1,4-diisopropylbenzene unit and has a non-planar kinked structure and a skewed structure. When this twisted structure monomer and the dianhydride constitute a polyimide chain, the novel diamine compound may exhibit ultra-low dielectric properties.

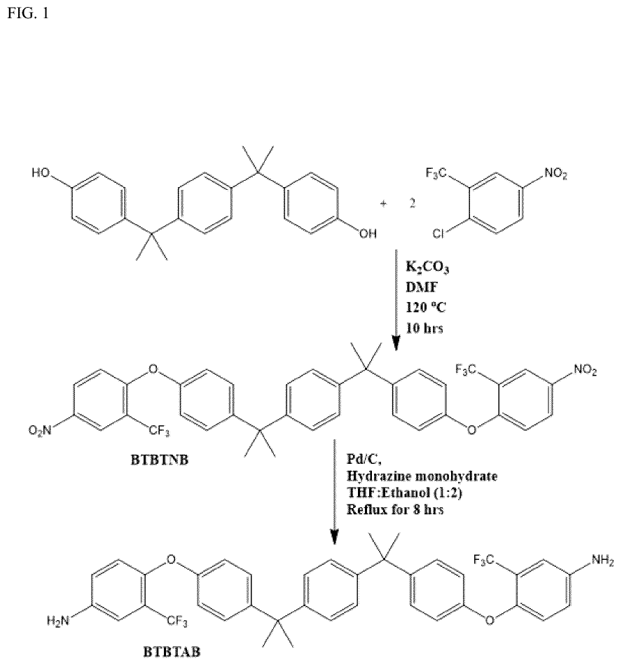

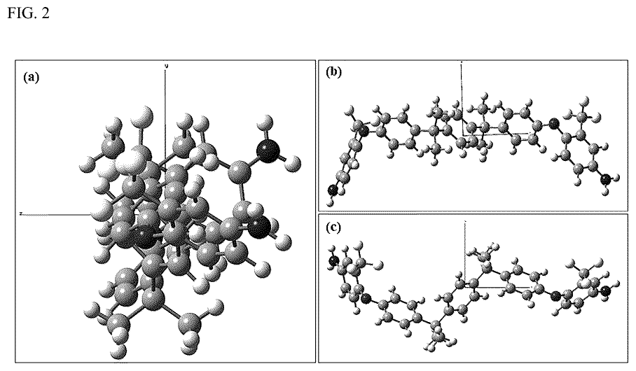

In one embodiment, the new diamine compound of the present disclosure may be synthesized through two synthetic procedures using bisphenol-P and 4-nitroaryl halide as starting materials, as shown in FIG. 1. In a first step, a dinitro compound (BTBTNB) may be synthesized and reduced to a new diamine compound (BTBTAB) through a hydrogenation reaction with a hydrazine compound as catalyzed by palladium/carbon (Pd/C).

In one embodiment, the novel diamine compound of the present disclosure may be used to prepare a polyimide precursor.

In another embodiment of the present disclosure, the polyimide precursor may be synthesized via a condensation polymerization reaction of a polymerization component including the diamine compound and one or more kinds of acid dianhydride. The polyimide precursor may be synthesized into polyimide via a thermal imidization process.

In one embodiment, the acid dianhydride that reacts with the diamine compound is not particularly limited, and may be preferably selected from pyromellitic dianhydride (PMDA), biphenyl-tetracarboxylic acid dianhydride (BPDA), 1,2,3,4-cyclobutanetetracarboxylic dianhydride (CBDA), and 4,4′-(hexafluoroisopropyliidene) diphthalic anhydride (6-FDA).

In an embodiment, the polyimide precursor may include a repeating unit represented by a Chemical Formula 2 as set forth below:

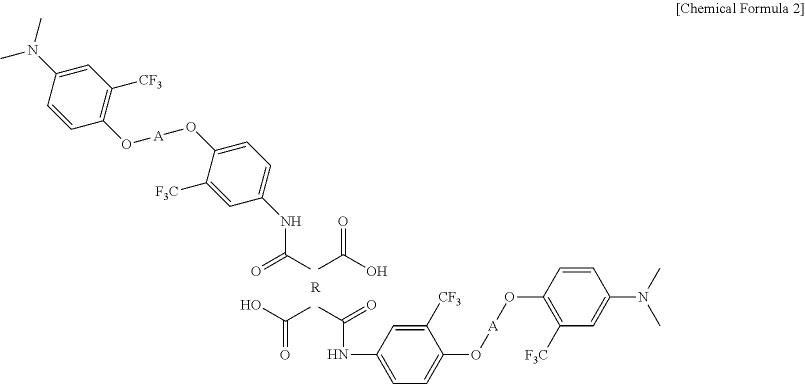

where in the Chemical Formula 2,

A is

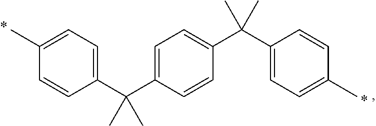

R is a tetravalent organic group,

* is a binding site.

In the Chemical Formula 2, the organic group of R refers to a substituent having four binding positions and includes all general organic functional groups. For example, the organic group of R is a tetravalent group with 6 to 22 carbon atoms including at least one aromatic ring.

In one embodiment, R may be selected from a group consisting of compounds respectively represented by Chemical Formulas 2-1 to 2-4 as set forth below:

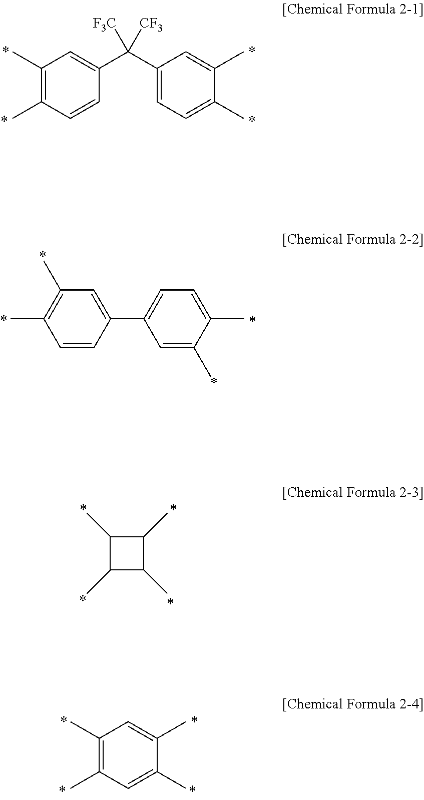

wherein * is a binding site.

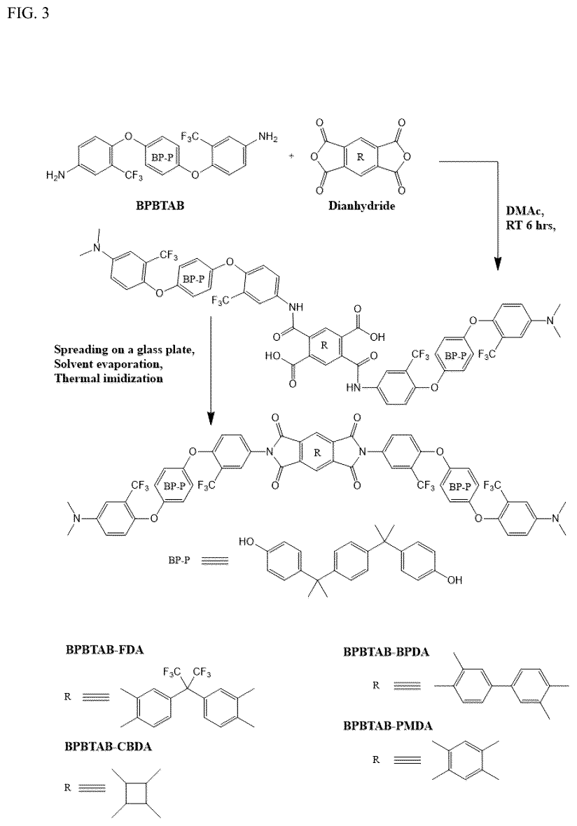

In one embodiment of the present disclosure, four types of polyimide (PI) films (BPBTAB-X) may be manufactured with acid dianhydrides such as PMDA, BPDA, CBDA, and 6-FDA via a two-step thermal imidization process as shown in FIG. 3. In the first step, the synthesized diamine is polymerized with dianhydride to produce each poly(amic acid) (PAAs), and a composition containing the poly(amic acid) may be applied on a substrate and then thermally imidized. In this regard, the composition containing the poly(amic acid) may be in a form of a solution. In this case, for example, when the polyimide precursor is synthesized in a solvent, the solution is either the obtained reaction solution itself, or is obtained by diluting this reaction solution with another solvent. Alternatively, when the polyimide precursor is obtained as a solid powder, the solid powder is dissolved in a solvent to produce the solution.

In one embodiment, the polyimide film may include a repeating unit represented by a Chemical Formula 3 as set forth below and may have a number average molecular weight (Mn) of about 10,000 to 40,000.

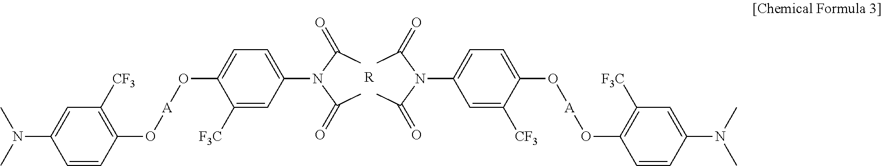

where in the Chemical Formula 3,

A is

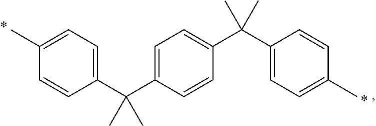

R is a tetravalent organic group,

* is a binding site.

In the Chemical Formula 3, the organic group of R refers to a substituent having four binding positions and includes all general organic functional groups. For example, the organic group of R is a tetravalent group with 6 to 22 carbon atoms including at least one aromatic ring.

In one embodiment, R may be selected from a group consisting of compounds respectively represented by Chemical Formulas 2-1 to 2-4 as set forth below:

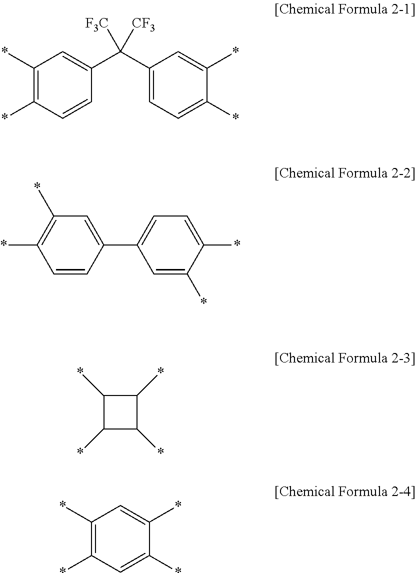 [SMILES: *c1ccc(C(c2ccc(*)c(*)c2)(C(F)(F)F)C(F)(F)F)cc1* ; *c1ccc(-c2ccc(*)c(*)c2)cc1* ; *C1C(*)C(*)C1* ; *c1cc(*)c(*)cc1*]

wherein * is a binding site.

The polyimide film synthesized according to the present disclosure has a low dielectric constant (Dₖ) of 2.11 or lower (1.13 to 1.84 at 1 MHz), a high 5% loss temperature (392 to 491° C.), and an appropriate glass transition temperature (Tg) (224 to 254° C.) and has a high tensile modulus (0.827 to 1.076 Gpa), thereby exhibiting excellent heat resistance and thermal stability. Furthermore, the polyimide film synthesized according to the present disclosure has excellent optical properties (T450 up to 83.37%), low moisture absorption (<0.63%), and especially excellent solubility in organic solvents.

As such, the polyimide film of the present disclosure has excellent thermal resistance and thermal stability, optical properties, low moisture absorption, and ultra-low dielectric properties. Thus, the polyimide film of the present disclosure may be applied to interlayer dielectric materials, wearable display devices, etc., and thus has high potential for application in 5G/6G communication applications and the microelectronics industry.

Hereinafter, examples of the present disclosure are described in detail. However, the examples as described as set forth below are only some embodiments of the present disclosure, and the scope of the present disclosure is not limited to the examples as set forth below.

### PRESENT EXAMPLE 1

Synthesis of 4,4′-(1,4-phenylenebis(propane-2,2diyl))diphenoxy-bis(2-(trifluoromethyl)4-nitrobenzene) (BPBTNB)

BPBTNB was synthesized via a reaction of 4-chloro-3(trifluoromethyl) nitrobenzene (4-CTNB) and bisphenol-P (BP-P) under presence of DMF solvent and K₂CO₃ at 120° C.

First, 5 g of BP-P is added to a 500 mL round flask and is dissolved in 20 g of anhydrous DMF to produce a BP-P solution. This solution is stirred at 120° C. for 30 minutes. In another vial, 8.65 g of 4-CTNB is dissolved in 10 mL of anhydrous DMF to produce a solution which in turn is added to the BP-P solution, thereby produce a mixture. This mixture is stirred at the same temperature for 10 hours. The mixture is cooled to room temperature and excess water is added thereto to precipitate a crude nitro compound. This crude mixture is recrystallized with EA/hexane (1:2) to obtain a pure nitro compound. A final product is obtained by drying the obtained pure nitro compound at 80° C. for 12 hours. (Yield: 93%, m.p: 219.1° C.)

Synthesis of 4,4′-(1,4-phenylenebis(propane-2,2-diyl))diphenoxy-bis(2-(trifluoromethyl)4-amino-benzene) (BPBTAB)

BPBTAB in accordance with the present disclosure was synthesized via hydrogenation reaction catalyzed by palladium/carbon (Pd/C). First, 5 g of BPBTNB and 0.5 g of palladium/carbon are added to a 250 mL round flask under a nitrogen atmosphere. A THF:ethanol mixture at a 2:1 ratio is added to the flask, and the mixture is subjected to reflux for 30 minutes, and then an appropriate amount of hydrazine monohydrate is carefully added to the flask for 30 minutes. This reaction is monitored using TLC for 8 hours and continuous reflux is performed. The reaction product is then filtered at high temperature over Celite 525catalyst in a Buchner funnel. The filtrate is cooled with crystalline amine and dried under vacuum at 80° C. for 15 hours to obtain BPBTAB. (See FIG. 1)

### PRESENT EXAMPLE 2

### Manufacturing of Polyimide (PI) Film

In accordance with the present disclosure, the poly(amic acid) was produced via ring-opening polymerization at room temperature and the PI film was synthesized via sequential thermal imidization. First, each of dianhydrides such as pyromellitic dianhydride (PMDA), biphenyl-tetracarboxylic acid dianhydride (BPDA), 1,2,3,4-cyclobutanetetracarboxylic dianhydride (CBDA), 6-FDA or FDA (4,4′-(hexafluoroisopropyliidene) diphthalic anhydride) was polymerized with diamine such that each of poly(amic acids) (PAAs) was prepared. PAA was then coated on a glass plate and thermally imidized at 220° C. under a nitrogen atmosphere (using a temperature program lamp: maintained for 3 hours at a temperature increase rate of 1.2° C. per minute to 80° C., and maintained at 180° C. for 1 hour, and maintained at 220° C. for 1 hour). The obtained PI film was removed from the glass plate by immersing the glass plate in double distilled water and then was dried at 100° C. to obtain the final dried film. FIG. 3 shows the synthesis route of the BTBTAB-X PI film based on the novel diamine BPBTAB and each of several commercial dianhydrides.

### COMPARATIVE EXAMPLE

Synthesis of α,α′-bis[4-(4-nitro-2-trifluoromethyl-phenoxy)phenyl]-1,3-diisopropylbenzene (BPBTNB-1)

First, 3.45 g of α,α′-bis(4-hydroxyphenyl)-1,3-diisopropylbenzene and 4.6 g of 2-chloro-5-nitrobenzotrifluoride are added to a 100 mL round flask and are dissolved in 15 mL of anhydrous DMF to produce a solution. Then, 3.44 g of calcium carbonate is added thereto, and the solution is stirred at 110° C. for 12 hours to produce a mixture. This mixture is cooled and precipitated with 70 mL of methanol to obtain a solid product. This solid product is washed sequentially with methanol and warm water, and then recrystallized with a DMF/methanol mixture to obtain 6.3 g of crystals. (Yield: 87%)

Synthesis of α,α′-bis[4-(4-amino-2-trifluoromethyl-phenoxy)phenyl]-1,3-diisopropylbenzene

6.3 g of dinitro compound (BPBTNB-1) is added to the flask in the presence of 0.1 g of palladium carbon and is heated at 70° C., then and 2.2 mL of hydrazine monohydrate is carefully added to the flask and then the reflux is performed for 2 hours. The Pd/C catalyst is removed therefrom, and a resulting product is condensed to a volume ratio of 1/3, and then cooled, such that white crystals are obtained. These white crystals are precipitated and dried under vacuum at room temperature to obtain 5.2 g of a final product.

### Manufacturing of Polyimide (PI) Film

A polyimide film was manufactured in the same manner as in Present Example 2, except that the diamine synthesized according to the Comparative Example was used.

### EXPERIMENTAL EXAMPLE 1

The synthesis of the dinitro compound BTBTNB and the new diamine compound BPBTAB in accordance with the present disclosure was identified based on FT-IR, ¹H-NMR, and ¹³C-NMR spectroscopic spectra. In the present disclosure, ¹H NMR and ¹³C NMR spectra were measured at Varian-400 (400 MHz) using d₆-DMSO as a solvent, and the FT-IR spectrum was measured using a Jasco-4100 IR spectrometer in a range of 400 to 4000 cm⁻¹ at a resolution of 4 cm⁻¹.

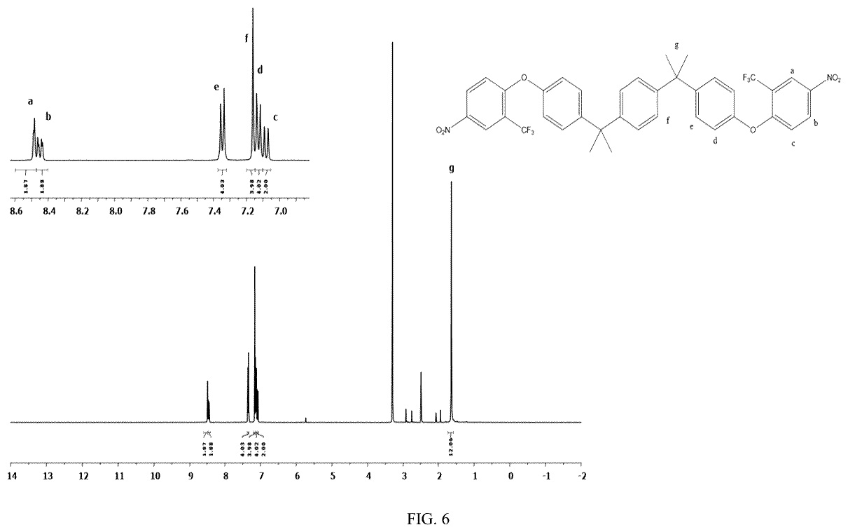

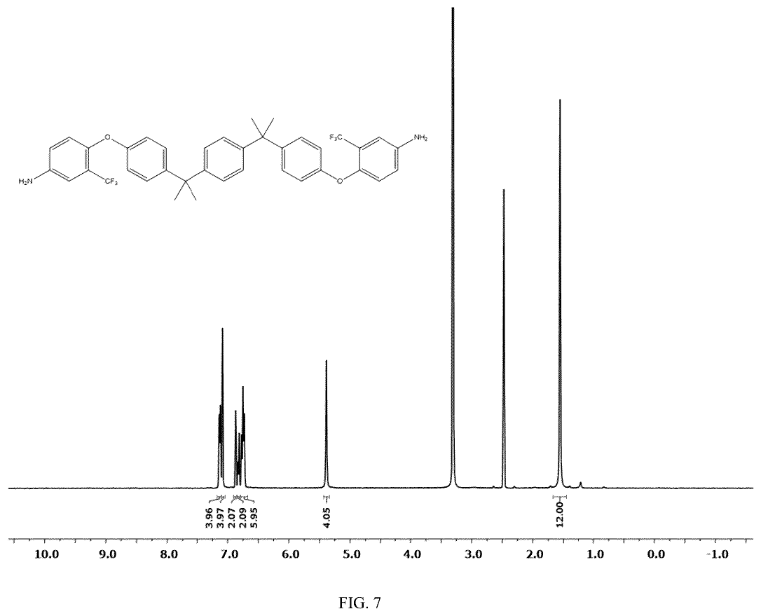

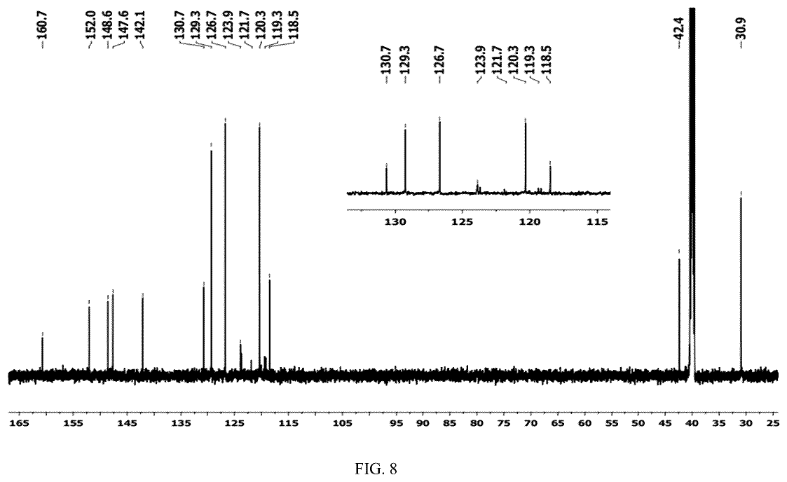

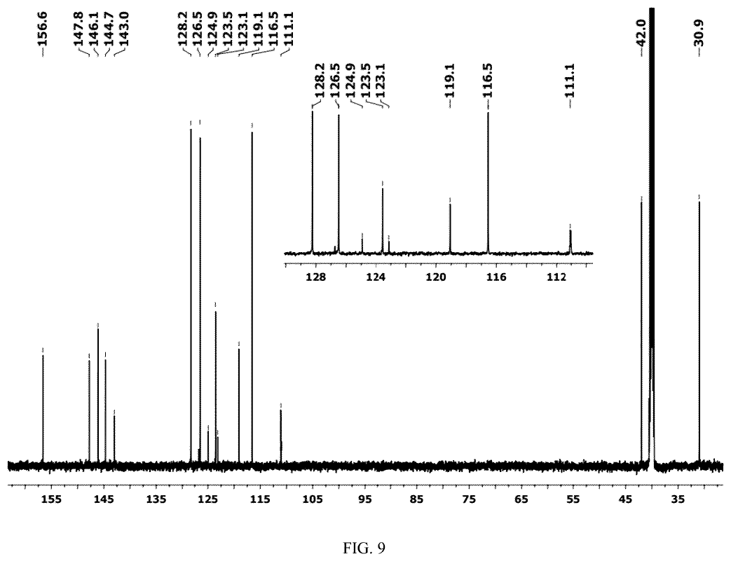

The data shown in FIGS. 5 to 9 were consistent with the structures of dinitro and diamine, and all signals were consistent with corresponding protons and carbons of the dinitro and diamine structures.

### EXPERIMENTAL EXAMPLE 2

A chemical structure of the polyimide film manufactured in accordance with the present disclosure was identified through FT-IR analysis.

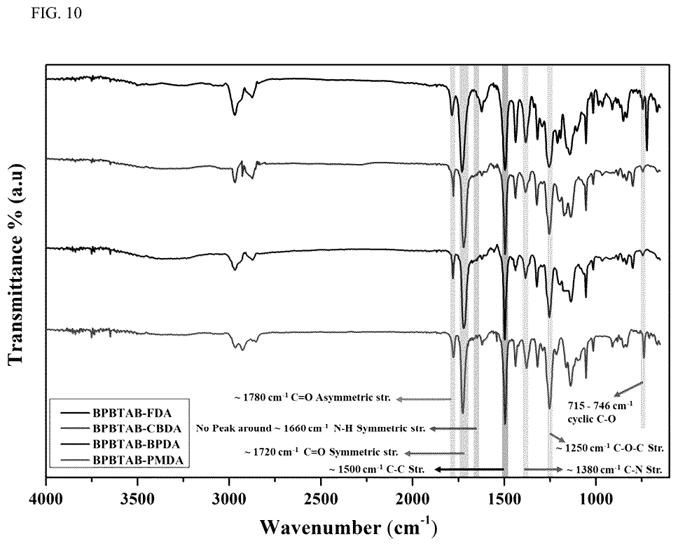

As shown in FIG. 10, the chemical structure of the PI film according to the Present Example of the present disclosure was characterized in the FT-IR spectral range of 4000 cm⁻¹ mid-IR range. All PI films exbibit infrared spectra with similar imide band characteristics approximately at 1760 to1785 cm⁻¹ (C═O asymmetric stretching), 1725 to 1745 cm⁻¹ (C═O symmetric stretching), 1360 to 1385 cm⁻¹ (CN stretching band), 740 to 745 cm⁻¹ (ring strain), and 680 to 720 cm⁻1 (C═O banding). This suggests the occurrence of the reaction between diamine and dianhydride and that the formation of the heterocyclic imide ring results in production of the poly(amic acid) (PAA). In addition, the average molecular weight (Mₙ, M_{w}, M_{z}) of PI was measured using GPC and is shown in a following Table 1. and this result shows that polyimide (PI) was successfully synthesized.

The number average molecular weight of the synthesized polyimide was in a range of 1.37×10⁴ to 4.09×10⁴, and the molecular weight distribution thereof was found to be in a range of 1.2 to 1.6.

TABLE 1

| | Mₙ × | M_{w} × | M_{z} × | | |
|---|---|---|---|---|---|
| | 10⁴ | 10⁴ | 10⁴ | | % F |
| | (g/mol) | (g/mol) | (g/mol) | PDI | content |
| BPBTAB-FDA | 4.09 | 6.09 | 9.35 | 1.5 | 20.7 |
| BPBTAB-CBDA | 2.44 | 3.05 | 3.62 | 1.2 | 13.4 |
| BPBTAB-BPDA | 1.37 | 2.17 | 2.88 | 1.6 | 12.0 |
| BPBTAB-PMDA | 2.42 | 3.14 | 3.95 | 1.3 | 13.0 |

### EXPERIMENTAL EXAMPLE 3

### Thermal and Mechanical Properties of PI Film

The thermal properties of the polyimide film in accordance with the present disclosure were investigated using thermogravimetric analysis (TGA), differential scanning calorimetry (DSC), and dynamic mechanical analysis (DMA). The mechanical properties thereof were investigated using the RB301 universal tester (RB301 UTM, Korea R&B).

In the present disclosure, each of the melting point of the monomer and the glass transition temperature (Tg) of the polyimide was measured using a Q-20 DSC instrument under heating of a nitrogenated atmosphere at 20° C./min and 10° C./min. Thermogravimetric measurements were conducted using a Q-50 TGA instrument at a heating rate of 20° C./min under a nitrogen flow rate of 20 mL/min. Tensile strength, tensile modulus, and elongation at break were calculated as a minimum value at a crosshead speed of 500 nm.

The measurement and calculation results may be identified in Table 2 as set forth below.

TABLE 2

| | | | | | Loss | | | |
|---|---|---|---|---|---|---|---|---|
| | TGA | Char | T_{g} | T_{g}** | modulus | Tensile | Tensile | Elongation |
| | T_{d}^{5%} | yield* | DSC | DMA | DMA | Strength | modulus | at break |
| | (° C.) | (%) | (° C.) | (° C.) | (MPa) | (MPa) | (GPa) | (%) |
| BPBTAB-FDA | 455 | 55.3 | 224 | 219 | 255 | 55.3 | 0.95 | 6.2 |
| BPBTAB- | 392 | 43.8 | 254 | 239 | 405 | 83.4 | 1.08 | 6.1 |
| CBDA | | | | | | | | |
| BPBTAB- | 491 | 60.7 | 236 | 210 | 226 | 85.9 | 0.83 | 6.7 |
| BPDA | | | | | | | | |
| BPBTAB- | 478 | 58.0 | 236 | 221 | 204 | 56.4 | 0.82 | 6.6 |
| PMDA | | | | | | | | |

Referring to Table 2, the synthesized polyimide film exhibited good thermal stability with almost no detectable loss up to 400° C. The 5% weight loss temperature (T_{d}^{5%}) of the PI film in accordance with the present disclosure is in a range of 392 to 491° C. with a maximum value of 491° C. Additionally, the PI film in accordance with the present disclosure exhibited a char yield of 60.7% at 800° C.

The glass transition temperature (Tg) of the PI film in accordance with the present disclosure was measured through DSC and compared with the glass transition temperature calculated from DMA measurement. As a result, all of the PI films according to the Present Example of the present disclosure exhibited appropriate glass transition temperature values (224 to 254° C.). However, the loss modulus value thereof reaches 204 MPa to 405 MPa. The glass transition temperature, the loss modulus, and the thermal stability of the PI film mainly depend on the strength and packing ability of the polyimide chain.

In general, the highly flexible BPBTAB-CBDA PI film exhibited the lowest T_{d}^{5%} (392° C.) and the highest loss modulus (405 MPa), while the BPBTAB-PMDA PI film exhibited the highest T_{d}^{5%} (478° C.) value and the highest loss modulus (204 MPa) and thus has better packing ability and rigidity structure compared to the other PI films.

Furthermore, based on the tensile property evaluation results of the PI films according to the Present Example of the present disclosure, all BPBTAB PI films in accordance with the present disclosure were cast as soft, flexible, transparent and tough films and had good physical properties. The tensile strength of these films ranges from 55.3 to 85.9 MPa. Additionally, the elongation at break and the tensile modulus thereof are closely related to each other and range from 6.1% to 6.7% and 0.82 to 1.08 GPa, respectively. Therefore, it is identified that the PI film exhibits appropriate thermal and mechanical properties for 5G/6G communication devices.

### Optical Properties of PI Film

The optical properties of the PI film in accordance with the present disclosure were measured in a 200 to 700 nm range using UV-Visible spectroscopy. The measurement results are shown in a following Table 3.

TABLE 3

| | (%) Transmittance at | | | CA |
|---|---|---|---|---|
| | 400 nm | 450 nm | 500 nm | (°) |
| BPBTAB-FDA | 46 | 83 | 85 | 95.73 |
| BPBTAB-CBDA | 71 | 79 | 81 | 99.76 |
| BPBTAB-BPDA | 0.8 | 78 | 85 | 96.65 |
| BPBTAB-PMDA | 0.3 | 20 | 76 | 100.17 |

As shown in Table 3, the transmittance depended on the fluorine content of the PI film. The fluorine reduces the interaction between the electrons accepting dianhydride and the electrons donating the diamine to reduce the CTC. Therefore, the BPTBB-FDA PI film with the highest fluorine content exhibits the highest optical transmittance (83% at 450 nm), followed by BPBTAB-CBDA PI (79%), BPBTAB-BPDA PI (78%), and BPBTAB-PMDA PI (20%). The optical properties depend on the structural feature of PI. In other words, the optical properties depend on the imide portion and the chain within or between CTCs formed via polymer chain packing of the PI film. As a result, the structural feature of the PI film that reduces the CTC improves the optical properties.

The 6F-isopropylidene and twisted isopropylidene group of the diamine portion interfere with intermolecular conjugation between PI chains, and 1,4-diisopropylbenzene unit interferes with interactions between PI chains. Additionally, the highly electronegative fluorine of the —CF₃ group hinders intrachain CTC by reducing the donating effect of the diamine. All these effects collectively reduce a λ₀ (wavelength) value and absorption at UV-Visible wavelengths.

### Evaluation of Moisture Absorption of PI Film

In the present disclosure, water absorbency of the PI film was evaluated via immersion of the PI film in water, and the evaluation result is shown in Table 4. Specifically, a water absorption test was conducted by immersing the PI film in deionized water at room temperature for about 24 hours. The moisture absorption was calculated based on a following equation (1).

$$Moisture absorption ( \% ) = \frac{{W}_{wet} - {W}_{dry}}{{W}_{dry}} \times 100 ( \% ) \qquad ( 1 )$$

W_{wet} denotes a weight of the PI film measured after immersion thereof in deionized water for about 24 hours, and Wary denotes the weight of the PI film measured after vacuum drying the PI film (100° C.) for about 24 hours.

TABLE 4

| | WU | CA |
|---|---|---|
| | (%) | (°) |
| BPBTAB-FDA | 0.23 | 95.73 |
| BPBTAB-CBDA | 0.25 | 99.76 |
| BPBTAB-BPDA | 0.38 | 96.65 |
| BPBTAB-PMDA | 0.63 | 100.17 |

As a result, it was confirmed that the WU % value of the PI film was very low (0.23% to 0.63%) and that the PI film of the present disclosure was much better than the Kapton R standard commercial PI film.

The BPBTAB-PMDA PI film exhibited the highest water absorption (0.63%), while the BPBTAB-FDA PI film with the highest fluorine concentration exhibited the lowest water absorption (0.23%). The —CF₃ group improves the hydrophobic characteristic of the PI film to lower the moisture absorption value of the PI film.

### Dielectric Properties of PI Film

A scheme for manufacturing the low-dielectric polyimide includes 1) a scheme of minimizing polarizability, 2) a scheme of providing a high free volume, and 3) a scheme of removing water to change the hydrophobicity of polyimide (PI), thereby changing the properties of diamine and dianhydride. The new monomer BPBTAB in accordance with the present disclosure may perform all of the schemes 1) to 3). The —CF₃ group not only increases the molar volume but also minimizes polarizability. Furthermore, the kinkable and skewed 1,4-diisopropylbenzene unit improve the molar volume and flexibility, thereby reducing the packing of polymer chains in the PI film.

In the present disclosure, the dielectric constant of the PI film was measured using a Keysight E4980L LCR meter (frequency range of 20 Hz to 1 MHz) at room temperature. Each copper adhesive sheet (7 μm thick) was disposed on each of both opposing surfaces of the PI film. Then, the capacitance of the PI film was measured. The relative permittivity (ε) of the PI film was calculated based on a following equation (2).

$${ε}_{r} = \frac{Cd}{{ε}_{0} A} \qquad ( 2 )$$

ε₀ denotes the vacuum dielectric constant (8.854×10⁻¹² F/m), ε and C denote the dielectric constant and capacitance of the PI film, respectively, and d and A denote the thickness and the area size of the PI film electrode, respectively.

TABLE 5

| | Dₖ at | | | D_{f} at | | |
|---|---|---|---|---|---|---|
| | 20 | 1 | 1 | 20 | 1 | 1 |
| | Hz | KHz | MHz | Hz | KHz | MHz |
| BPBTAB-FDA | 1.37 | 1.33 | 1.13 | 0.0097 | 0.0184 | 0.0542 |
| BPBTAB-CBDA | 1.47 | 1.45 | 1.28 | 0.0078 | 0.0128 | 0.0443 |
| BPBTAB-BPDA | 1.83 | 1.81 | 1.59 | 0.0088 | 0.0132 | 0.0454 |
| BPBTAB-PMDA | 2.11 | 2.07 | 1.84 | 0.0069 | 0.0127 | 0.0420 |
| Comparative | 2.51 | 2.41 | 2.38 | — | — | — |
| Example (FDA) | | | | | | |

The Dₖ and D_{f} of the BPBTAB-based PI film in accordance with the present disclosure are presented in the above Table 5 and range between 1.13 and 2.11, which is much smaller than that of the commercial PI Kapton R (Dₖ=3.2-3.4), and which satisfies a condition for high-frequency 5G/6G communications. This value is much smaller than that of the linear bisphenol-A based monomer, which has a Dk value of 2.87. Furthermore, the dielectric constant value of the monomer-FDA based polyimide film as the Comparative Example was in a range of 2.38 to 2.51. Thus, the PI film in accordance with the present disclosure exhibited a much lower dielectric constant value than that of the polyimide film according to the Comparative Example.

In the present disclosure, the kinked and skewed PI backbone increases the characteristics of the bulky and bent BPBTAB monomer which suppresses the close intermolecular packing of BPBTAB PI chains and increases the interchain free volume, resulting in a significant reduction in the Dₖ value. When the size of the monomer is larger, the number of polar imide groups per unit volume is reduced, thereby suppressing the dipolar polarization, which leads to a low dielectric constant.

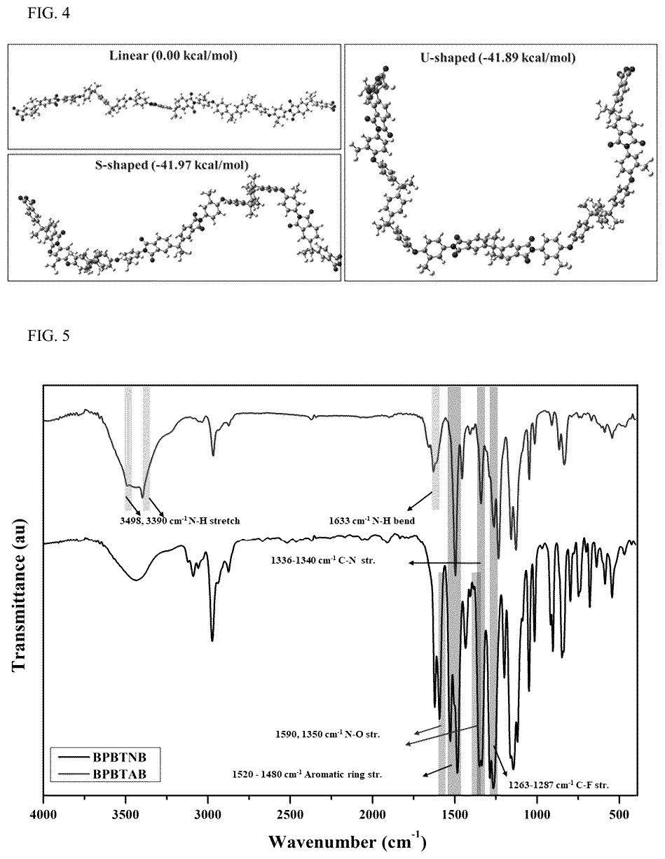

Referring to the 3D structure of FIG. 4 of the BPBTAB-FDA PI in accordance with the present disclosure, the 3D structure has three different structures: linear, U-, and S-conformation. Non-planar kinked and skewed structures such as U- or S-shapes are more stable than the linear structure, and in particular, the S-shaped structure has a conformational energy of −41.97 kcal/mol and is the most stable. The excellent transmittance and high-performance dielectric properties of BPBTAB-FDA PI are due to this stability.

The PI film has high-performance dielectric properties due to the combination of the effect of the kinkable/skew structure as shown in FIG. 4 and the effect of fluorine, such that the Dₖ value decreases to a maximum, thus reaching 1.13 at 1 MHz. The PI based on PMDA and BPDA in accordance with the present disclosure has the polarizability higher than that of each of BPBTAB-CBDA PI and BPBTAB-FDA PI, and has a kind of CT interaction and exhibits a higher Dₖ value than that of each of BPBTAB-CBDA and BPBTAB-FDA. The dielectric loss factor (D_{f}) of the PI based on PMDA and BPDA in accordance with the present disclosure exhibits a value of 0.04 to 0.05 based on 1 MHz.

### Solubility of Polyimide (PI)

The qualitative solubility of polyimide (PI) at a concentration of 20 mg per mL was investigated at room temperature and in a state heated to 60° C., and the results are shown in Table 6 as set forth below. In this regard, +++ means being soluble at room temperature, ++− means being soluble under heating (60° C.), +−− means being partially soluble under heating (60° C.), −− means being insoluble under heating (60° C.).

TABLE 6

| PI | | | | |
|---|---|---|---|---|
| Solvent | BPBTAB-FDA | BPBTAB-CBDA | BPBTAB-BPDA | BPBTAB-PMDA |
| DMF | + + + | + + + | + + − | + + − |
| DMAc | + + + | + + + | + + − | + + − |
| DMSO | + + + | + + + | + + + | + + + |
| NMP | + + + | + + + | + + + | + + + |
| γ-Butyrolactone | + + + | + + + | + − − | + − − |
| THF | + + + | + + − | + + − | + + − |
| Toluene | + + + | + − − | + − − | + − − |
| Xylene | + + + | + − − | + − − | + − − |
| Chloroform | + + + | + − − | + + − | + + − |
| DCM | + + + | + − − | + + − | + − − |
| Ethyl acetate | + + − | + + − | + − − | + − − |

As a result, the FDA-based PI in accordance with the present disclosure has high solubility in polar solvents such as DMF, DMAc, DMSO, NMP, GBL, and THF and non-polar solvents such as toluene, cresol, chloroform, and DCM. Good solubility of PI means good material processability. BPDA- and PMDA-based PIs in accordance with the present disclosure have lower solubility in both polar and non-polar solvents. However, CBDA-based PI exhibited good solubility in polar solvents, but poor solubility in non-polar solvents.

The FDA-based PI in accordance with the present disclosure has high solubility in polar and nonpolar solvents, as shown in Table 6, because a portion between the chains is more flexible, the packing is loose, and the main chain has many bulky CF₃ groups and CH₃ groups.

Although the embodiments of the present disclosure have been described above with reference to the accompanying drawings, the present disclosure may not be limited to the embodiments and may be implemented in various different forms. Those of ordinary skill in the technical field to which the present disclosure belongs will be able to appreciate that the present disclosure may be implemented in other specific forms without changing the technical idea or essential features of the present disclosure. Therefore, it should be understood that the embodiments as described above are not restrictive but illustrative in all respects.

What is claimed is:

1. A diamine compound represented by Chemical Formula 1 as set forth below:

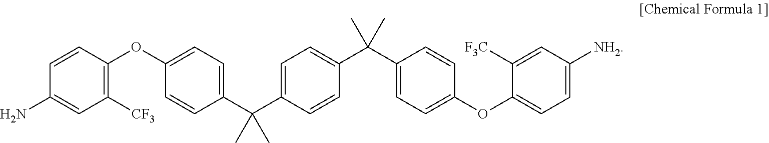 [SMILES: CC(C)(c1ccc(Oc2ccc(N)cc2C(F)(F)F)cc1)c1ccc(C(C)(C)c2ccc(Oc3ccc(N)cc3C(F)(F)F)cc2)cc1]

2. The diamine compound of claim 1, wherein the diamine compound is used for preparing a polyimide precursor.

3. A polyimide precursor prepared by polymerizing a polymerization component including the diamine compound according to claim 1 and at least one kind of acid dianhydride.

4. The polyimide precursor of claim 3, wherein the acid dianhydride is selected from pyromellitic dianhydride (PMDA), biphenyl-tetracarboxylic acid dianhydride (BPDA), 1,2,3,4-cyclobutanetetracarboxylic dianhydride (CBDA), and 4,4′-(hexafluoroisopropyliidene) diphthalic anhydride (6-FDA).

5. The polyimide precursor of claim 3, wherein the polyimide precursor includes a repeating unit represented by a following Chemical Formula 2: 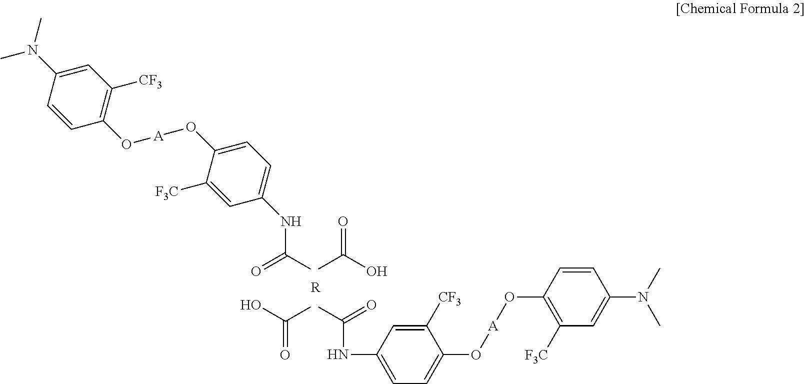
where in the Chemical Formula 2,
A is 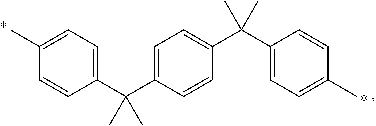
R is a tetravalent organic group.
* is a binding site.

6. The polyimide precursor of claim 5, wherein R is selected from a group consisting of compounds respectively represented by following Chemical Formulas 2-1 to 2-4: 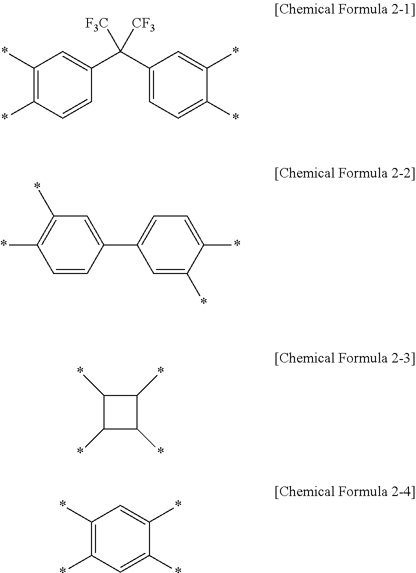 [SMILES: *c1ccc(C(c2ccc(*)c(*)c2)(C(F)(F)F)C(F)(F)F)cc1* ; *c1ccc(-c2ccc(*)c(*)c2)cc1* ; *C1C(*)C(*)C1* ; *c1cc(*)c(*)cc1*]
wherein * is a binding site.

7. A polyimide film manufactured by applying a composition containing the polyimide precursor according to claim 3 on a substrate and then thermally imidizing the applied composition.

8. The polyimide film of claim 7, wherein the polyimide film includes a repeating unit represented by a following Chemical Formula 3: 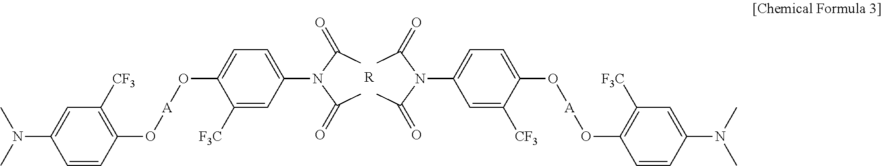
where in the Chemical Formula 3,
A is 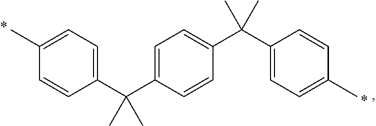
R is a tetravalent organic group.
* is a binding site.

9. The polyimide film of claim 8, wherein R is selected from a group consisting of compounds respectively represented by following Chemical Formulas 2-1 to 2-4: 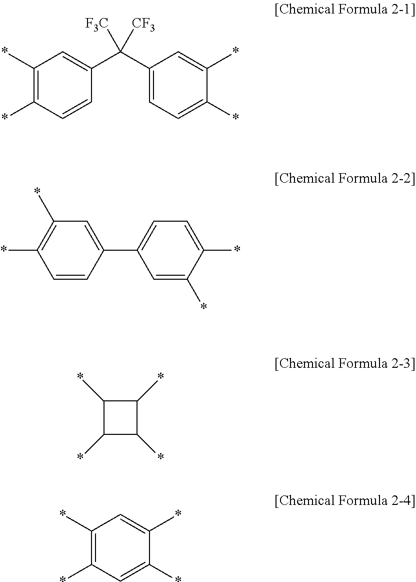 [SMILES: *c1ccc(C(c2ccc(*)c(*)c2)(C(F)(F)F)C(F)(F)F)cc1* ; *c1ccc(-c2ccc(*)c(*)c2)cc1* ; *C1C(*)C(*)C1* ; *c1cc(*)c(*)cc1*]
wherein * is a binding site.

10. The polyimide film of claim 7, wherein the polyimide film has a dielectric constant (Dₖ) of 2.11 or lower.
